(19) United States
(12) Patent Application Publication
(10) Pub. No.: US 2026/0166258 A1
(43) Pub. Date: Jun. 18, 2026

(54) METHOD AND SYSTEM FOR INDUCING COMFORTABLE AWAKENING IN SLEEP STATE

(71) Applicant: SB Solutions Inc., Ulsan (KR)

(72) Inventor: Seungup SEO, Ulsan (KR)

(73) Assignee: SB Solutions Inc., Ulsan (KR)

(21) Appl. No.: 19/405,176

(22) Filed: Dec. 1, 2025

Related U.S. Application Data
(63) Continuation of application No. PCT/KR2024/005411, filed on Apr. 22, 2024.

(30) Foreign Application Priority Data
May 31, 2023 (KR) 10-2023-0070084

(51) Int. Cl. A61M 21/00

(52) U.S. Cl. CPC A61M 21/00; A61M 2021/0022; A61M 2021/0083; A61M 2205/3375; A61M 2230/42; A61M 2230/63

(57) ABSTRACT

A method and system for inducing comfortable awakening in a sleep state are disclosed. The system for inducing awakening according to one embodiment may comprise: a sensor device attached to a body of a user and configured to collect first data comprising at least one of movement data of the user and respiration data of the user and transmit the collected first data to a user device, and configured to generate vibration having a pattern corresponding to a control signal received from the user device, and the user device configured to collect second data comprising sound information related to sleep of the user, determine a sleep stage of the user by analyzing a sleep cycle of the user using the first data received from the sensor device and the second data, and transmit, to the sensor device, the control signal for generating vibration having a pattern determined based on the determined sleep stage and a preset awakening time.

### CROSS-REFERENCE TO RELATED APPLICATIONS

This U.S. non-provisional application is a continuation of PCT International Application No. PCT/KR 2024/005411, filed on Apr. 22, 2024, which claims priority under 35 U.S.C. 119 to Korean Patent Application No. 10-2023-0070084, filed with the Korean Intellectual Property Office on May 31, 2023. The disclosures of the foregoing applications are incorporated herein by reference in their entirety.

### BACKGROUND OF THE INVENTION

In general awakening, waking up by hearing a loud alarm sound may cause various adverse effects on the body and may instead induce insomnia in which sleep is disturbed. In addition, when a user is forcibly awakened during sleep due to an alarm sound, not only may hormonal imbalance occur, but the brain may also be subjected to stress. As stress hormones well known in the art, such as adrenaline, epinephrine, and cortisol, are secreted, the sympathetic nervous system is excited, such that blood pressure increases to a level higher than that at a usual time of awakening, heart rate increases, and blood glucose level also increases. When the sympathetic nervous system is repeatedly extremely activated due to being startled awake by a loud alarm sound, physical responses such as sharp increases in blood pressure and blood glucose and tension may become chronic, and risks of developing cardiovascular disease, diabetes, metabolic syndrome, and depression may be increased.

In some cases, there are users who are unable to awaken from sleep at once after hearing an alarm and thus set the alarm to sound multiple times. However, such a habit is more detrimental to health because it increases sleep inertia. Sleep inertia refers to a state in which a person is unable to fully escape from drowsiness, and is generally intensified when a person is abruptly awakened from deep sleep, which is commonly referred to as stage 3 sleep. Sleep is divided into rapid eye movement (REM) sleep, in which dreaming occurs, and non-rapid eye movement (NREM) sleep, in which dreaming does not occur, and the NREM sleep is further divided into stage 1 sleep and stage 2 sleep, which are light sleep stages, and stage 3 sleep (or stage 3 and stage 4 sleep). In the morning, the likelihood of being in stage 3 sleep (or stage 3 and stage 4 sleep) is generally very low; however, when a person is repeatedly awakened by an alarm and then falls asleep again, secretion of adenosine, which is a sleep hormone that induces deep sleep, remains in an active state. This leads to chronic fatigue. If a state of grogginess continues, it is preferable to compensate even with a short nap.

### BRIEF SUMMARY OF THE INVENTION

The present invention provides a method and system for inducing awakening capable of inducing awakening of a user by vibration according to a sleep stage of the user based on an analyzed sleep cycle of the user and a preset awakening time.

The present invention also provides a system for inducing awakening comprising: a sensor device attached to a body of the user, configured to collect first data comprising at least one of movement data and respiration data of the user and transmit the collected first data to a user device, and configured to generate vibration having a pattern corresponding to a control signal received from the user device; and the user device configured to collect second data comprising sound information related to sleep of the user, determine a sleep stage of the user by analyzing a sleep cycle of the user using the first data received from the sensor device and the second data, and transmit, to the sensor device, the control signal for generating vibration having a pattern determined based on the determined sleep stage and the preset awakening time.

According to one aspect, the sensor device may be configured to generate the vibration according to the pattern by adjusting, for a predetermined period of time, at least one of whether to generate the vibration and a magnitude of the vibration according to the pattern.

According to another aspect, the respiration data may be generated by continuously measuring, based on at least one of a change in a resonant frequency generated through an oscillator and repetitive charging and discharging of the sensor, a change in a fringing field formed through a sensor comprised in the sensor device and attached to the body of the user, according to respiration activity of the user.

According to still another aspect, the second data may comprise: at least one of a respiration sound of the user, a snoring sound of the user, and a sound generated according to movement of the user during sleep, which are collected through a microphone comprised in the user device.

A sensor device attached to a body of a user, comprising: a sensing unit configured to collect first data comprising at least one of movement data of the user and respiration data of the user, and second data comprising sound information related to sleep of the user; a control unit configured to determine a sleep stage of the user by analyzing a sleep cycle of the user using the first data and the second data, and generate a control signal for generating vibration having a pattern determined based on the determined sleep stage and a preset awakening time; and an output unit configured to generate vibration having a pattern corresponding to the control signal.

According to one aspect, the control unit may be configured to generate the vibration according to the pattern by adjusting, through the output unit and for a predetermined period of time, at least one of whether to generate the vibration and a magnitude of the vibration according to the pattern.

According to another aspect, the sensing unit may comprise: a sensor attached to the body of the user; and a measurement circuit configured to measure sensing data of the sensor, wherein the measurement circuit may be configured to generate the respiration data by continuously measuring, based on at least one of a change in a resonant frequency generated through an oscillator and repetitive charging and discharging of the sensor, a change in a fringing field (fringing field) formed through the sensor according to respiration activity of the user.

According to still another aspect, the sensing unit may comprise: a microphone, and the second data may comprise at least one of a respiration sound of the user, a snoring sound of the user, and a sound generated according to movement of the user during sleep, which are collected through the microphone.

A method for inducing awakening of a sensor device attached to a body of a user, comprising collecting first data comprising at least one of movement data of the user and respiration data of the user; transmitting the first data to a user device through a communication unit comprised in the sensor device; receiving a control signal from the user device through the communication unit; and generating vibration having a pattern corresponding to the control signal through an output unit comprised in the sensor device, wherein the user device collects second data comprising sound information related to sleep of the user, determines a sleep stage of the user by analyzing a sleep cycle of the user using the first data and the second data, and generates and transmits, to the sensor device, the control signal for generating vibration having a pattern determined based on the determined sleep stage and a preset awakening time.

A method for inducing awakening of a sensor device attached to a body of a user, comprising collecting first data comprising at least one of movement data of the user and respiration data of the user; collecting second data comprising sound information related to sleep of the user through a microphone comprised in the sensor device; under control of a control unit comprised in the sensor device, determining a sleep stage of the user by analyzing a sleep cycle of the user using the first data and the second data; under control of the control unit, determining a vibration pattern based on the determined sleep stage and a preset awakening time; and generating vibration according to the determined pattern through an output unit comprised in the sensor device.

By analyzing a sleep cycle of a user, awakening of the user may be induced by vibration according to a sleep stage of the user corresponding to the analyzed sleep cycle and a preset awakening time.

### BRIEF DESCRIPTION OF THE DRAWINGS

FIG. 1 is a diagram illustrating an example of an overall configuration of a system for inducing awakening according to one embodiment of the present invention.

FIG. 2 is a diagram illustrating an example of an awakening induction process according to a sleep stage determined through a sleep cycle in one embodiment of the present invention.

FIG. 3 is a diagram illustrating an example of changes in a sensor device according to respiration activity of a user in one embodiment of the present invention.

FIG. 4 is a diagram illustrating an example of a fringing field in one embodiment of the present invention.

FIG. 5 is a diagram illustrating an example of an internal configuration of a sensor device according to one embodiment of the present invention.

FIG. 6 is a diagram illustrating an example of a measurement circuit according to one embodiment of the present invention.

FIG. 7 is a diagram illustrating an example of an operation of a clock counter in one embodiment of the present invention.

FIG. 8 is a diagram illustrating another example of the measurement circuit according to one embodiment of the present invention.

FIG. 9 is a diagram illustrating an example of an operation of an ADC in one embodiment of the present invention.

FIG. 10 is a flowchart illustrating an example of an awakening induction method according to one embodiment of the present invention.

FIG. 11 is a flowchart illustrating another example of the awakening induction method according to one embodiment of the present invention.

FIG. 12 is a block diagram illustrating an example of a computer device according to one embodiment of the present invention.

### DETAILED DESCRIPTION OF THE INVENTION

Hereinafter, embodiments will be described in detail with reference to the accompanying drawings. However, various changes may be made to the embodiments, and thus the claims of the patent application are not limited or limited by these embodiments. It should be understood that all changes, equivalents, and alternatives to the embodiments are included in the claims.

Terms used in the embodiments are used for the purpose of description only, and should not be construed as limiting. The singular forms “a”, “an”, and “the” include plural forms unless the context clearly dictates otherwise. It should be understood that the terms “comprises”, “comprising”, “includes”, “including”, “having” and the like in this specification specify the presence of stated features, numbers, steps, operations, components, parts or combinations thereof, but do not preclude the presence or addition of one or more other features, numbers, step, operations, components or combinations thereof.

Unless otherwise defined, all terms used herein, including technical or scientific terms, have the same meaning as commonly understood by one of ordinary skill in the art to which the embodiments belong. Terms, such as those defined in commonly used dictionaries, should be interpreted as having a meaning that is consistent with their meaning in the context of the relevant art and will not be interpreted in an idealized or overly formal sense unless expressly so defined herein.

Furthermore, in the description with reference to the accompanying drawings, the same components are given the same reference numerals regardless of the drawing reference numerals, and redundant descriptions thereof will be omitted. In describing the embodiments, when it is determined that detailed descriptions of related known technologies may unnecessarily obscure the gist of the embodiments, such detailed descriptions will be omitted.

Furthermore, in describing components of the embodiments, terms such as first, second, A, B, (a), and (b) may be used. Such terms are used only for the purpose of distinguishing one component from another and do not limit the nature, order, or sequence of the corresponding components. When a component is described as being “connected,” “coupled,” or “linked” to another component, it should be understood that the component may be directly connected, coupled, or linked to the other component, or one or more other components may be connected, coupled, or linked between the respective components.

Components included in any one embodiment and components having common functions will be described using the same names in other embodiments. Unless otherwise stated, descriptions provided in any one embodiment may be applied to other embodiments, and detailed descriptions will be omitted in overlapping ranges.

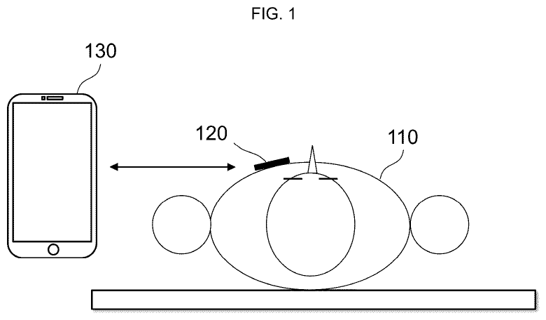

FIG. 1 is a diagram illustrating an example of an overall configuration of a system for inducing awakening according to one embodiment of the present invention. FIG. 1 illustrates an example of a user 110, a sensor device 120 attached to a body of the user 110, and a user device 130 of the user 110. The system for inducing awakening according to one embodiment may include the sensor device 120 and the user device 130.

The sensor device 120 may be attached to a specific site of the user, such as a thorax of the user 110, but is not limited thereto. The sensor device 120 may collect first data related to movement and/or respiration of the user 110, and may wirelessly communicate with the user device 130. For example, the sensor device 120 may transmit the collected first data of the user 110 to the user device 130. The first data collected by the sensor device 120 may include movement data and/or respiration data. As an example, the sensor device 120 may include an accelerometer and/or a gyroscope, and may collect output of the accelerometer and/or the gyroscope included in the sensor device 120 as the movement data. As another example, the sensor device 120 may collect respiration data, such as a respiration pattern and/or a respiration cycle of the user 110, by continuously measuring changes according to respiration activity of the user 110. Collection of such respiration data will be described in more detail later.

The user device 130 may collect second data. For example, the user device 130 may collect the second data through a sound signal input through a microphone. As an example, the second data may include a respiration sound of the user 110, a snoring sound, and/or a sound generated according to movement of the user 110 during sleep.

In this case, the user device 130 may determine a sleep stage of the user 110 by analyzing a sleep cycle of the user 110 based on the first data collected and transmitted by the sensor device 120 and the second data collected by the user device 130.

In addition, the user device 130 may induce the user 110 to comfortably awaken from a sleep state by vibration appropriate for the sleep stage of the user 110, based on the determined sleep stage of the user 110 and an awakening time (or alarm time) set by the user 110 in the user device 130.

Vibration may be transmitted to the user 110 through the sensor device 120. To this end, the user device 130 may transmit a control signal to the sensor device 120 to provide vibration corresponding to a sleep stage of the user 110.

To this end, a computer program as an application for an awakening induction service may be installed and executed in the user device 130, and the user device 130 may operate under control of such a computer program.

In addition, according to an embodiment, the sensor device 120 may include functions of the user device 130. In this case, the system for inducing awakening may include the sensor device 120. For example, the sensor device 120 may include a microphone and further collect second data, such as a respiration sound of the user 110, a snoring sound, and a sound generated according to movement of the user 110 during sleep.

In this case, the sensor device 120 may determine a sleep stage of the user 110 by analyzing a sleep cycle of the user 110 based on the first data and the second data collected by the sensor device 120. In addition, the sensor device 120 may induce the user 110 to comfortably awaken from a sleep state by vibration corresponding to the sleep stage of the user 110, based on the determined sleep stage of the user 110 and an awakening time (or alarm time) set by the user 110.

Inducing awakening of the user 110 by vibration may be performed by generating a vibration pattern according to a sleep stage of the user 110 from a predetermined time prior to an awakening time (or alarm time) set by the user 110. For example, when a sleep state of the user 110 is a stage 3 sleep state (or a stage 4 sleep state), the sensor device 120 may generate vibration having a preset pattern according to the stage 3 sleep state (or the stage 4 sleep state) from 10 minutes prior to the awakening time (or alarm time) set by the user 110, thereby inducing the user 110 to gradually transition to an awakened state and helping the user 110 to awaken refreshingly.

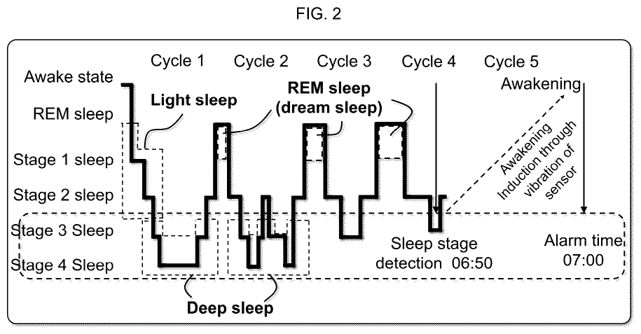

FIG. 2 is a diagram illustrating an example of an awakening induction process according to a sleep stage determined through a sleep cycle in one embodiment of the present invention. A general sleep cycle maintains about five cycles, and stage 1 sleep and REM sleep have brain wave patterns very similar to those of an awakened state. In other words, a state of the brain is close to the awakened state. When a user is awakened during such a sleep state, a time required for the brain to adjust to the awakened state is short, there is no groggy feeling, and a mood may be refreshed. In contrast, stage 3 sleep and stage 4 sleep are states of deep sleep. When the user 110 is abruptly awakened from such states, a long time is required to transition to the awakened state, and thus the user 110 may experience a groggy feeling for a period of time.

The system for inducing awakening according to embodiments of the present invention may analyze a sleep cycle of the user 110 based on first data (movement data and respiration data of the user 110) and second data (respiration sound of the user 110, snoring sound, and sounds generated according to movement of the user 110 during sleep, etc.) collected through the sensor device 120 and/or the user device 130. Thereafter, the system for inducing awakening may induce awakening of the user 110 by vibration corresponding to a sleep stage of the user through the sensor device 120 from several minutes to several tens of minutes prior to an awakening time (in the embodiment of FIG. 2, from 10 minutes prior according to a stage 3 sleep state or a stage 4 sleep state), according to the sleep stage of the user 110 determined as a result of the analysis and the awakening time (or alarm time) set by the user 110. At this time, an intensity and a pattern of the vibration may vary according to a sleep state of the user 110 and settings of the user 110. Accordingly, the system for inducing awakening may help the user 110 to awaken refreshingly by gradually transitioning the user 110 from a deep sleep state to an awakened state.

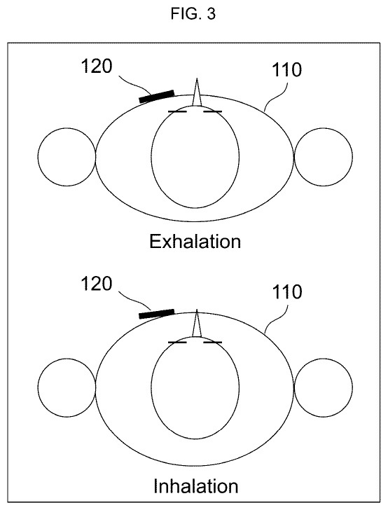

FIG. 3 is a diagram illustrating an example of changes in a sensor device according to respiration activity of a user in one embodiment of the present invention. FIG. 3 illustrates an example of a sensor device 120 attached to a user 110 performing respiration activity. A site such as a thorax of the user 110 undergoes a change in volume and generates movement according to respiration activity of the user 110.

The sensor device 120 may be attached to such a specific site of the user 110. At this time, the sensor device 120 may be attached so as not to be completely in close contact with an outer surface of the user 110. For example, in the case of a human body, the sensor device 120 may be attached such that only a part of one surface of the sensor device 120 is attached to skin of the human body, so that the entire corresponding surface of the sensor device 120 is not in close contact with the skin of the human body.

At this time, when the user 110 performs respiration activity, movement occurs as a volume of a thorax changes, and according to such movement, a degree of contact between the sensor device 120 and an outer surface of the user 110 continuously changes, thereby inducing a constant variation. In the embodiment of FIG. 3, it is illustrated that a degree of contact between the sensor device 120 and the user 110 changes during inhalation and exhalation of the user 110.

At this time, the sensor device 120 may measure respiration data, such as a respiration pattern and/or a respiration cycle of the user 110, by continuously measuring such changes according to respiration activity of the user 110.

In one embodiment, the sensor device 120 may form a fringing field that penetrates into an interior of a surface of the user 110 by using two or more electrodes. According to an embodiment, the fringing field may be formed to reach at least the surface of the user 110. At this time, the sensor device 120 may obtain information on respiration of the user 110 by measuring changes in the fringing field according to respiration activity of the user 110. In this case, as a method of measuring changes in the fringing field according to respiration activity of the user 110, an oscillator and/or repetitive charging and discharging of the sensor device 120 may be utilized.

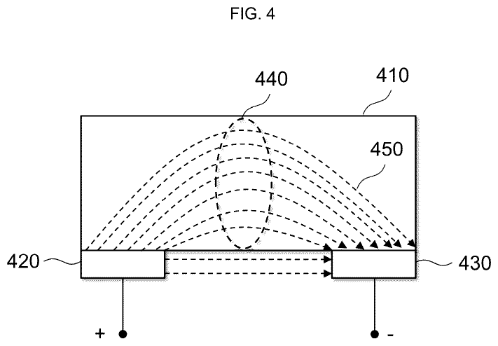

FIG. 4 is a diagram illustrating an example of a fringing field in one embodiment of the present invention. FIG. 4 illustrates two electrodes 420, 430 attached to a MUT (Material Under Test, 410). In this case, as a voltage is applied to the two electrodes 420, 430, a fringing field 440 may be formed into the interior of the MUT 410 between the two electrodes 420, 430, as illustrated in FIG. 4.

In FIG. 4, the fringing field (440 is illustrated as a dotted ellipse for ease of understanding; however, in practice, the fringing field 440 may be formed by electromagnetic force lines (for example, field lines 450 of FIG. 4) between two conductors when a voltage is biased to a capacitor.

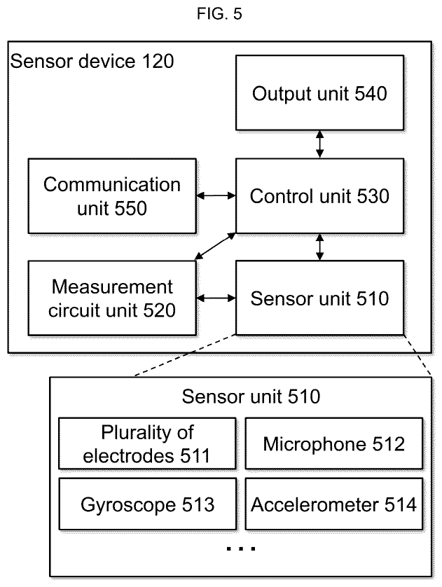

FIG. 5 is a diagram illustrating an example of an internal configuration of a sensor device according to one embodiment of the present invention. The sensor device 120 according to the embodiment of FIG. 5 may include a sensing unit 510, a measurement circuit 520, a control unit 530, an output unit 540, and a communication unit 550.

As illustrated in FIG. 5, the sensing unit 510 may include various sensors, such as a plurality of electrodes 511, a microphone 512, a gyroscope 513, and/or an accelerometer 514.

Here, the plurality of electrodes 511 may include the two electrodes (420, 430 for forming the fringing field (440 described above with reference to FIG. 4, and may be used to obtain respiration data of the user 110, which may be included in the first data.

In addition, the microphone 512 may be included to collect second data, such as a respiration sound of the user 110, a snoring sound, and sounds generated according to movement of the user 110 during sleep, in an embodiment in which the user device 130 is not used.

The gyroscope 513 and/or the accelerometer 514 may be used to obtain movement data of the user 110, which may be included in the first data.

Components of the sensing unit 510 may vary according to embodiments, such that at least some components may be omitted or new components may be added. For example, in order to obtain environmental information related to a sleep cycle of the user 110, the sensing unit 510 may further include a temperature sensor or a humidity sensor.

The measurement circuit 520 may include a measurement circuit for reading sensor data (or sensing data) through the sensing unit 510. For example, the measurement circuit 520 may read respiration data based on the plurality of electrodes 511. The measurement circuit 520 will be described in more detail later.

The control unit 530 may control operations of the sensing unit 510, the measurement circuit 520, and the output unit 540, and may control the communication unit 550 to transmit measured data to the user device 130 or to receive setting values (for example, an awakening time (or alarm time)) from the user device 130. The communication unit 550 may include a communication module for a wired or wireless connection (preferably a wireless connection) with the user device 130. Data communication between the communication unit 550 and the user device 130 may be performed using at least one of various well-known communication protocols, such as BLE (Bluetooth Low Energy), NFC (Near Field Communication), WiFi, and the like.

The output unit 540 may output vibration having a specific pattern under control of the control unit 530. For example, the output unit 540 may include a motor, and the control unit 530 may control the motor to control the output unit 540 such that vibration having a specific pattern is output. Here, generating vibration having a specific pattern may mean that at least one of whether to generate vibration and a magnitude of the vibration is adjusted over a predetermined period of time.

With regard to respiration data, the user device 130 may be a user terminal such as a smartphone or a smart watch. For example, with regard to respiration data, the user device 130 may display data collected by the sensor device 120 (for example, waveform data), and may display a respiration rate of the user 110, a quality of respiration, a quality of sleep, and the like, which are determined based on the collected data. To this end, an algorithm for determining the respiration rate, the quality of respiration, the quality of sleep, and the like may be executed in the sensor device 120 or the user device 130. When the algorithm is executed in the sensor device 120, the sensor device 120 may further transmit, to the user device 130, information on the respiration rate of the user 110, the quality of respiration, the quality of sleep, and the like determined using the collected data, in addition to the collected data.

As one embodiment, in a case where the system for inducing awakening includes both the sensor device 120 and the user device 130, the sensor device 120 may be attached to a body of the user 110 and may collect first data (movement data and/or respiration data) of the user 110 using the sensing unit 510 and/or the measurement circuit 520 under control of the control unit 530. In this case, the sensor device 120 may transmit the collected first data to the user device 130 through the communication unit 550 under control of the control unit 530.

Meanwhile, the user device 130 may collect second data including sound information related to sleep of the user 110. At this time, the user device 130 may analyze a sleep cycle of the user 110 using the first data received from the sensor device 120 and the second data collected by the user device 130. In addition, the user device 130 may determine a sleep stage of the user according to the analyzed sleep cycle. In addition, the user device 130 may determine a vibration pattern based on the determined sleep stage and an awakening time (or alarm time) set in the user device 130 by the user. At this time, the user device 130 may generate a control signal for generating vibration having the determined pattern and transmit the control signal to the sensor device 120.

In this case, the sensor device 120 may induce comfortable awakening of the user 110 by outputting, through the output unit 540, vibration having a pattern corresponding to a control signal received from the user device 130 under control of the control unit 530, thereby providing vibration corresponding to a sleep stage of the user 110.

As another embodiment, in a case where the system for inducing awakening induces awakening of the user 110 without including the user device 130 and includes the sensor device 120, the sensor device 120 may collect first data (movement data and/or respiration data) and/or second data (respiration sound, snoring sound, and/or sounds generated according to movement of the user 110 during sleep, etc.) using the sensing unit 510 and/or the measurement circuit 520. In this case, the sensor device 120 may analyze a sleep cycle of the user 110 based on the first data and/or the second data collected through the control unit 530, and may determine a sleep stage of the user 110 according to the analyzed sleep cycle. Thereafter, the control unit 530 of the sensor device 120 may determine a vibration pattern based on the sleep stage of the user 110 and an awakening time set by the user 110, and may control the output unit 540 to generate vibration according to the determined pattern.

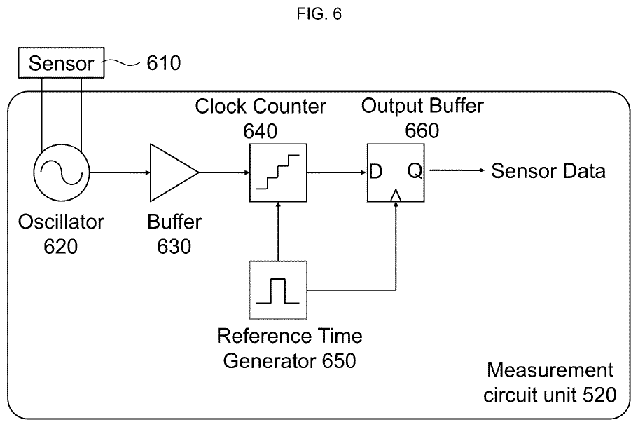

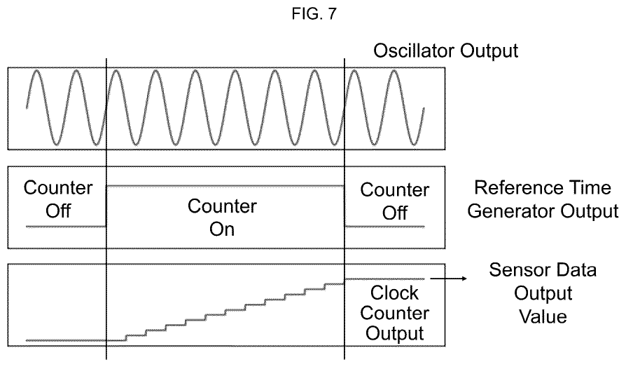

FIG. 6 is a diagram illustrating an example of a measurement circuit according to one embodiment of the present invention, and FIG. 7 is a diagram illustrating an example of an operation of a clock counter in one embodiment of the present invention. The measurement circuit 520 according to the embodiment of FIG. 6 may include a circuit for measuring respiration data.

The measurement circuit 520 may include an oscillator 620 connected to a sensor 610, a buffer 630, a clock counter 640, a reference time generator 650, and an output buffer 660.

The sensor 610 may correspond to the plurality of electrodes 511 described above. For example, as a voltage is applied to at least two electrodes (for example, the two electrodes 420, 430) included in the sensor 610, a fringing field may be formed. The oscillator 620 may be an RC (Resistor-Capacitor) oscillator or an LC (Inductor-Capacitor) oscillator. At this time, when the fringing field changes according to respiration, an output frequency (resonant frequency) of the oscillator 620 connected to the sensor 610 may change. In this case, an output signal of the oscillator 620 may be input to the clock counter 640 through the buffer 630.

The clock counter 640 may count cycles of an input signal during a reference time of the reference time generator 650. Since a greater number of cycles may be counted during the reference time as a frequency of the input signal increases, an output value of the clock counter 640 may increase. The reference time generator 650 may generate a reference time signal during which the clock counter 640 operates.

An output of the clock counter 640 may be output as sensor data through the output buffer 660.

FIG. 7 illustrates an example in which, when an output of the oscillator 620 (a signal of a resonant frequency) is input to the clock counter 640, the clock counter 640 counts cycles of the output of the oscillator 620 according to an output of the reference time generator 650 and outputs the counted value as an output value of the sensor data.

As described above, a fringing field formed through the sensor 610 may change according to respiration of the user 110, an output resonant frequency of the oscillator 620 may change according to the change in the fringing field, and an output value of the clock counter 640 may change according to the change in the resonant frequency. Accordingly, information on respiration of the user 110 (respiration data) may be obtained based on changes in the output value of the clock counter 640.

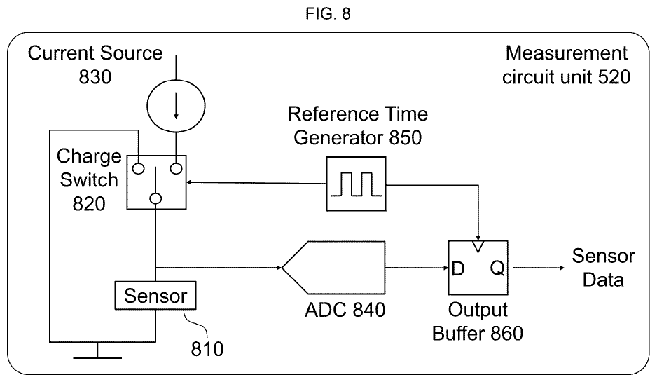

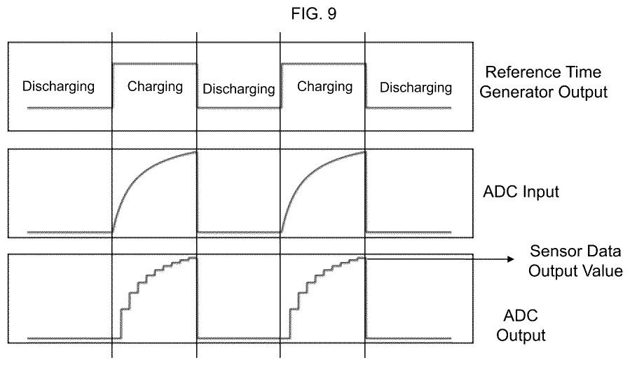

FIG. 8 is a diagram illustrating another example of a measurement circuit according to one embodiment of the present invention, and FIG. 9 is a diagram illustrating an example of an operation of an ADC in one embodiment of the present invention.

The measurement circuit 520 according to the embodiment of FIG. 8 may include a charge switch 820, a current source 830, an ADC 840, a reference time generator 850, and an output buffer 860 connected to a sensor 810.

The sensor 810 may correspond to the plurality of electrodes 511 described above. In the embodiment of FIG. 8, the sensor 810 is illustrated as being included in the measurement circuit 520; however, in practice, the sensor 810 may be disposed outside the measurement circuit 520 so as to be attached to the user 110.

The measurement circuit 520 may measure a degree of charging by repeatedly charging and discharging the sensor 810 using the charge switch 820. The reference time generator 850 may generate a control signal having a reference time interval to operate the charge switch 820. When the charge switch 820 is turned on, the sensor 810 and the current source 830 may be connected such that the sensor 810 is charged, and when the charge switch 820 is turned off, the connection between the sensor 810 and the current source 830 may be released such that the sensor 810 is discharged.

While the sensor 810 is being charged, a voltage at an input terminal of the sensor 810 may increase, and the measurement circuit 520 may convert the voltage into a digital code using the ADC 840. At this time, an output value of the ADC 840 at a time point when the charge switch 820 is turned off by the reference time generator 850 may be output as sensor data through the output buffer 860.

FIG. 9 illustrates inputs and outputs of the ADC 840 according to repeated connection and disconnection between the sensor 810 and the current source 830 by the charge switch 820 according to an output of the reference time generator 850. In addition, FIG. 9 illustrates that an output value of the ADC 840 at a time point when the charge switch 820 is turned off by the reference time generator 850 may be output as a sensor data output value.

As described above, as a fringing field changes according to respiration of the user 110, a degree to which the sensor 810 is charged may change, and an output value of the ADC 840 may change according to the change in the degree to which the sensor 810 is charged. Accordingly, information on respiration of the user 110 (respiration data) may be obtained based on changes in the output value of the ADC 840.

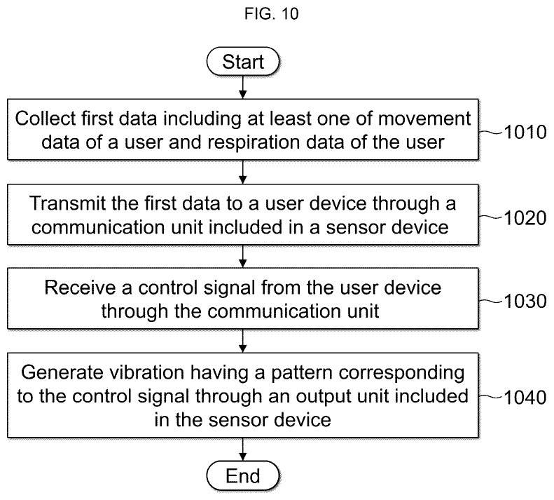

FIG. 10 is a flowchart illustrating an example of a method for inducing awakening according to one embodiment of the present invention. The method for inducing awakening according to the present embodiment may be performed by the sensor device 120 described above. As one embodiment, the control unit 530 of the sensor device 120 may include at least one processor and a memory. In this case, an operation of the sensor device 120 may be interpreted as being implemented by the processor of the control unit 530 controlling the sensing unit 510, the measurement circuit 520, and the communication unit 550 included in the sensor device 120 according to code of a computer program stored in the memory of the control unit 530.

In step 1010, the sensor device 120 may collect first data including at least one of movement data and respiration data of the user 110. As one embodiment, the sensor device 120 may include a sensing unit configured to collect first data including at least one of movement data and respiration data of the user 110. The sensing unit herein may include the sensing unit 510 and the measurement circuit 520 described above with reference to FIG. 5. In this case, the sensing unit may include, as a part of the sensing unit 510, a sensor attached to a body of the user 110 (for example, the sensor 610 and/or the sensor 810), and the measurement circuit 520 may measure sensing data of the sensor. In this case, the measurement circuit 520 may generate respiration data by continuously measuring, based on a change in a resonant frequency generated through an oscillator or repetitive charging and discharging of the sensor, a change in a fringing field formed through the sensor according to respiration activity of the user 110. The movement data of the user 110 may include an output of an accelerometer and/or a gyroscope, as described above.

In step 1020, the sensor device 120 may transmit the first data to the user device 130 through the communication unit 550 included in the sensor device 120. At this time, the user device 130 may collect second data including sound information related to sleep of the user 110, determine a sleep stage of the user 110 by analyzing a sleep cycle of the user 110 using the first data and the second data, and generate and transmit, to the sensor device 120, a control signal for generating vibration having a pattern determined based on the determined sleep stage and a preset awakening time. Here, the second data may include at least one of a respiration sound of the user 110, a snoring sound of the user 110, and a sound generated according to movement of the user 110 during sleep, which are collected through a microphone included in the user device 130, and the awakening time may be preset by the user 110.

In step 1030, the sensor device 120 may receive the control signal from the user device 130 through the communication unit 550.

In step 1040, the sensor device 120 may generate vibration having a pattern corresponding to the control signal through the output unit 540 included in the sensor device 120. To this end, the control unit 530 included in the sensor device 120 may generate the vibration according to the pattern by adjusting, through the output unit 540 and for a predetermined period of time, at least one of whether to generate the vibration and a magnitude of the vibration according to the pattern.

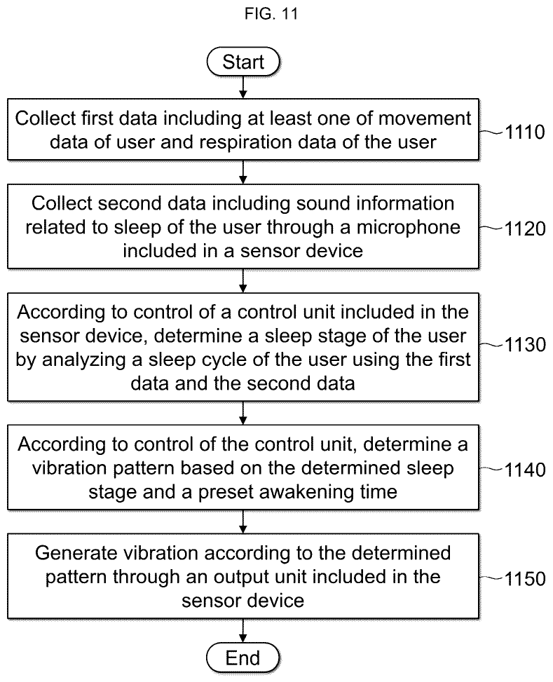

FIG. 11 is a flowchart illustrating another example of a method for inducing awakening according to one embodiment of the present invention. The method for inducing awakening according to the present embodiment may be performed by the sensor device 120 described above. As one embodiment, the control unit 530 of the sensor device 120 may include at least one processor and a memory. In this case, an operation of the sensor device 120 may be interpreted as being implemented by the processor of the control unit 530 controlling the sensing unit 510, the measurement circuit 520, and the communication unit 550 included in the sensor device 120 according to code of a computer program stored in the memory of the control unit 530.

In step 1110, the sensor device 120 may collect first data including at least one of movement data of the user and respiration data of the user. As described above, in one embodiment, the sensor device 120 may include a sensing unit configured to collect first data including at least one of movement data and respiration data of the user 110. The sensing unit herein may include the sensing unit 510 and the measurement circuit 520 described above with reference to FIG. 5. In this case, the sensing unit may include, as a part of the sensing unit 510, a sensor attached to a body of the user 110 (for example, the sensor 610 and/or the sensor 810), and the measurement circuit 520 may measure sensing data of the sensor. In this case, the measurement circuit 520 may generate respiration data by continuously measuring, based on a change in a resonant frequency generated through an oscillator or repetitive charging and discharging of the sensor, a change in a fringing field formed through the sensor according to respiration activity of the user 110. The movement data of the user 110 may include an output of an accelerometer and/or a gyroscope, as described above.

In step 1120, the sensor device 120 may collect second data including sound information related to sleep of the user 110 through a microphone 512 included in the sensor device 120. Here, the second data may include at least one of a respiration sound of the user 110, a snoring sound of the user 110, and a sound generated according to movement of the user 110 during sleep, which are collected through the microphone 512, and an awakening time may be preset by the user 110. For example, the sensor device 120 may receive a setting value of the awakening time through the communication unit 550 and store the setting value.

In step 1130, the sensor device 120 may determine a sleep stage of the user 110 by analyzing a sleep cycle of the user 110 using the first data and the second data under control of the control unit 530 included in the sensor device 120. The sleep stage of the user 110 may include, as described above with reference to FIG. 2, five stages including a REM sleep stage and stages 1 through 4 as NREM sleep stages, but is not limited thereto. For example, stage 3 and stage 4 may be used as a single integrated stage.

In step 1140, the sensor device 120 may determine a vibration pattern based on the determined sleep stage and the preset awakening time under control of the control unit 530. Patterns empirically determined for each determined sleep stage of the user 110 may be preset. In addition, time information indicating how long before the awakening time each pattern should start may be included in each of the patterns.

In step 1150, the sensor device 120 may generate vibration according to the determined pattern through the output unit 540 included in the sensor device 120. In this case, the control unit 530 included in the sensor device 120 may generate vibration according to the pattern by adjusting, through the output unit 540 and for a predetermined period of time, at least one of whether to generate vibration and a magnitude of the vibration according to the pattern. For example, the control unit 530 may induce awakening of the user 110 by generating vibration according to the determined pattern from a predetermined time prior to a preset awakening time according to time information included in the pattern.

Meanwhile, the user device 130 may be implemented by, for example, a computer device.

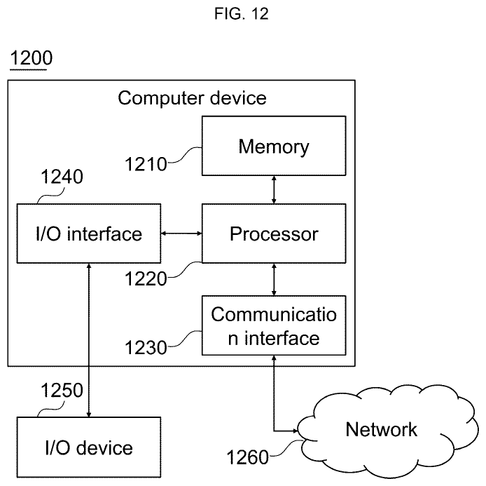

FIG. 12 is a block diagram illustrating an example of a computer device according to one embodiment of the present invention. As illustrated in FIG. 12, a computer device 1200 may include a memory 1210, a processor 1220, a communication interface 1230, and an I/O interface, 1240. The memory 1210 is a computer-readable recording medium and may include non-volatile mass storage devices such as RAM (random access memory), ROM (read only memory), and a disk drive. Here, non-volatile mass storage devices such as the ROM and the disk drive may be included in the computer device 1200 as separate permanent storage devices distinguished from the memory 1210. In addition, the memory 1210 may store an operating system and at least one program code. Such software components may be loaded into the memory 1210 from a computer-readable recording medium separate from the memory 1210. Such separate computer-readable recording media may include computer-readable recording media such as a floppy drive, a disk, a tape, a DVD/CD-ROM drive, and a memory card. In another embodiment, the software components may be loaded into the memory 1210 through the communication interface 1230 rather than through a computer-readable recording medium. For example, the software components may be loaded into the memory 1210 of the computer device 1200 based on a computer program installed by files received through a network 1260.

The processor 1220 may be configured to process instructions of a computer program by performing basic arithmetic, logic, and input/output operations. The instructions may be provided to the processor 1220 by the memory 1210 or the communication interface 1230. For example, the processor 1220 may be configured to execute instructions received according to program code stored in a recording device such as the memory 1210.

The communication interface 1230 may provide a function for the computer device 1200 to communicate with other devices through the network 1260. For example, requests, commands, data, files, and the like generated by the processor 1220 of the computer device 1200 according to program code stored in a recording device such as the memory 1210 may be transmitted to other devices through the network 1260 under control of the communication interface 1230. Conversely, signals, commands, data, files, and the like from other devices may be received by the computer device 1200 through the communication interface 1230 of the computer device 1200 via the network 1260. Signals, commands, data, and the like received through the communication interface 1230 may be delivered to the processor 1220 or the memory 1210, and files and the like may be stored in a storage medium (the above-described permanent storage device) that may be further included in the computer device 1200.

The I/O interface 1240 may be a means for interfacing with an I/O device 1250. For example, input devices may include devices such as a microphone, a keyboard, or a mouse, and output devices may include devices such as a display or a speaker. As another example, the input/output interface 1240 may be a means for interfacing with a device in which input and output functions are integrated into a single device, such as a touchscreen. The input/output device 1250 may be configured as a single device together with the computer device 1200.

In other embodiments, the computer device 1200 may include fewer or more components than those illustrated in FIG. 12. However, it is not necessary to clearly illustrate most conventional components. For example, the computer device 1200 may be implemented to include at least some of the above-described input/output devices 1250, or may further include other components such as a transceiver or a database.

As such, according to embodiments of the present invention, a sleep cycle of a user may be analyzed, and awakening of the user may be induced by vibration according to a sleep stage of the user corresponding to the analyzed sleep cycle and a preset awakening time.

The system or apparatus described above may be implemented using hardware components, or a combination of hardware components and software components. For example, the apparatuses and components described in the embodiments may be implemented using one or more general-purpose computers or special-purpose computers, such as a processor, a controller, an ALU (arithmetic logic unit), a DSP (digital signal processor), a microcomputer, an FPGA (field programmable gate array), a PLU (programmable logic unit), a microprocessor, or any other device capable of executing and responding to instructions. A processing device may execute an OS (operating system) and one or more software applications executed on the operating system. In addition, the processing device may access, store, manipulate, process, and generate data in response to execution of software. For convenience of understanding, a single processing device may be described as being used; however, those skilled in the art will appreciate that the processing device may include a plurality of processing elements and/or a plurality of types of processing elements. For example, the processing device may include a plurality of processors, or a single processor and a single controller. In addition, other processing configurations, such as a parallel processor, are also possible.

Software may include a computer program, code, instructions, or a combination of one or more thereof, and may configure a processing device to operate as desired or independently or collectively instruct the processing device. Software and/or data may be embodied in any type of machine, component, physical device, virtual equipment, computer storage medium, or device in order to be interpreted by a processing device or to provide instructions or data to the processing device. Software may be distributed over network-connected computer systems and stored or executed in a distributed manner. Software and data may be stored in one or more computer-readable recording media.

A method according to an embodiment may be implemented in the form of program instructions executable through various computer means and recorded on a computer-readable medium. The computer-readable medium may include program instructions, data files, data structures, or a combination thereof. The medium may continuously store an executable program, or temporarily store the program for execution or download. In addition, the medium may be various recording means or storage means in a form in which a single or multiple hardware components are combined, and is not limited to a medium directly connected to a computer system, but may be distributed and present over a network. Examples of the medium may include magnetic media such as hard disks, floppy disks, and magnetic tapes, optical recording media such as CD-ROMs and DVDs, magneto-optical media such as floptical disks, and media configured to store program instructions, such as ROMs and flash memories. In addition, other examples of the medium may include recording media or storage media managed by app stores distributing applications or by sites or servers supplying or distributing various types of software. Examples of the program instructions may include not only machine code generated by a compiler, but also high-level language code executable by a computer using an interpreter or the like.

Although the embodiments have been described above with reference to limited embodiments and drawings, those skilled in the art will appreciate that various modifications and variations may be made based on the above description. For example, the described techniques may be performed in an order different from the described methods, and/or components of the described systems, structures, apparatuses, circuits, and the like may be combined or assembled in a form different from the described methods, or may be replaced or substituted with other components or equivalents, while still achieving appropriate results.

Accordingly, other implementations, other embodiments, and equivalents to the claims also fall within the scope of the claims set forth below.

1. A system for inducing awakening, comprising:
a sensor device attached to a body of a user and configured to collect first data comprising at least one of movement data of the user and respiration data of the user and transmit the collected first data to a user device, and configured to generate vibration having a pattern corresponding to a control signal received from the user device; and
the user device configured to collect second data comprising sound information related to sleep of the user, determine a sleep stage of the user by analyzing a sleep cycle of the user using the first data received from the sensor device and the second data, and transmit, to the sensor device, the control signal for generating vibration having a pattern determined based on the determined sleep stage and a preset awakening time,
wherein the respiration data is generated by continuously measuring, based on at least one of a change in a resonant frequency generated through an oscillator and repetitive charging and discharging of a sensor comprised in the sensor device and attached to the body of the user, a change in a fringing field (fringing field) formed through the sensor according to respiration activity of the user.

2. The system for inducing awakening of claim 1, wherein:
the sensor device generates the vibration according to the pattern by adjusting, for a predetermined period of time, at least one of whether to generate the vibration and a magnitude of the vibration according to the pattern.

3. The system for inducing awakening of claim 1, wherein:
the second data comprises at least one of a respiration sound of the user, a snoring sound of the user, and a sound generated according to movement of the user during sleep, which are collected through a microphone comprised in the user device.

4. A sensor device attached to a body of a user, comprising:
a sensing unit configured to collect first data comprising at least one of movement data of the user and respiration data of the user and second data comprising sound information related to sleep of the user;
a control unit configured to determine a sleep stage of the user by analyzing a sleep cycle of the user using the first data and the second data, and generate a control signal for generating vibration having a pattern determined based on the determined sleep stage and a preset awakening time; and
an output unit configured to generate vibration having a pattern corresponding to the control signal,
wherein the sensing unit comprises:
a sensor attached to the body of the user; and
a measurement circuit configured to measure sensing data of the sensor, and
wherein the measurement circuit generates the respiration data by continuously measuring, based on at least one of a change in a resonant frequency generated through an oscillator and repetitive charging and discharging of the sensor, a change in a fringing field (fringing field) formed through the sensor according to respiration activity of the user.

5. The sensor device of claim 4, wherein:
the control unit generates the vibration according to the pattern by adjusting, through the output unit and for a predetermined period of time, at least one of whether to generate the vibration and a magnitude of the vibration according to the pattern.

6. The sensor device of claim 4, wherein:
the sensing unit comprises a microphone, and
the second data comprises at least one of a respiration sound of the user, a snoring sound of the user, and a sound generated according to movement of the user during sleep, which are collected through the microphone.

7. A method for inducing awakening performed by a sensor device attached to a body of a user, comprising:
collecting first data comprising at least one of movement data of the user and respiration data of the user;
transmitting the first data to a user device through a communication unit comprised in the sensor device;
receiving a control signal from the user device through the communication unit; and
generating vibration having a pattern corresponding to the control signal through an output unit comprised in the sensor device,
wherein the user device collects second data comprising sound information related to sleep of the user, determines a sleep stage of the user by analyzing a sleep cycle of the user using the first data and the second data, and generates and transmits, to the sensor device, the control signal for generating vibration having a pattern determined based on the determined sleep stage and a preset awakening time, and
the respiration data is continuously measured based on at least one of a change in a resonant frequency generated through an oscillator and repetitive charging and discharging of a sensor comprised in the sensor device and attached to the body of the user, by measuring a change in a fringing field (fringing field) formed through the sensor according to respiration activity of the user.
